(19) United States
(12) Patent Application Publication
(10) Pub. No.: US 2011/0275907 A1
(43) Pub. Date: Nov. 10, 2011

(54) Electronic Health Journal

(76) Inventors: Salvatore Richard Inciardi, Manalapan, NJ (US); Sandra D. Elliott, Barnegat, NJ (US)

(21) Appl. No.: 13/042,130

(22) Filed: Mar. 7, 2011

Related U.S. Application Data
(60) Provisional application No. 61/332,325, filed on May 7, 2010.

(51) Int. Cl. A61B 5/00

(52) U.S. Cl. 600/301

(57) ABSTRACT

A system for creating an electronic health journal comprising: at least one sensor configured to monitor an objective physiological parameter of a user and generate signals indicative of the monitored objective physiological parameter; a mobile electronic device comprising: input means for entering information relating to a subjective physiological condition of the user; an internal memory device configured to store the information relating to the subjective physiological condition of the user and the signals indicative of the monitored objective physiological parameter as a data log; and a transmitter; a local base station; and a remote data store, wherein the local base station is configured to automatically receive the data log from the mobile electronic device upon the mobile electronic device coming into communication proximity with the local base station and wherein the local base station is configured to transmit the data log to the remote data store.

### CROSS-REFERENCE TO RELATED PATENT APPLICATION

The present patent application claims the benefit of United Stated Provisional Patent Application Ser. No. 61/332,325, filed May 7, 2010, the entirety of which is hereby incorporated by reference.

### FIELD OF THE INVENTION

This invention relates generally to systems for remotely monitoring a variety of patient data in connection with a health care provider or facility, and more particularly, to an electronic health journal for collecting data with the goal of maximizing patient comfort, convenience and optimizing the delivery of patient care and patient services within institutions as well as within the community. The monitoring of patient data can also encompass data that may not necessarily be considered within typical clinical care or patient care parameters and can include data which is supportive of health and wellness activities such as diet tracking and weigh management.

### BACKGROUND OF THE INVENTION

An electronic health journal is a portable device that provides patient data collection of various health variables and comfort measures for use by the patient, typically a consumer. Patient data collected from the device can be stored locally within the device until it can be downloaded to a remote repository or to a central data repository. Such data may be downloaded from the device through a near field communication or NFC reader which can be tethered to a computer, an embedded reader within cell phone handsets, so-called “smart phones” and similar personal communication or personal digital assistant type devices (collectively “PDAs”). Typically, PDAs include some type of interface, usually a screen for entering data, a memory for data storage and at least one of the following for connectivity: IrDA, Bluetooth, NFC and/or Wi-Fi. However, some PDAs (typically those used primarily as telephones) may not have a touch screen, using softkeys, a directional pad and either the numeric keypad or a thumb keyboard for input. Like Bluetooth, NFC is a short-range high frequency wireless communication technology which enables the exchange of data between devices over relatively close distances. Patient data downloaded via the computer and stored locally can then be sent to a central data repository via the internet or through cellular networks for data collected via cell phone handsets and/or PDAs. The data will be collected and displayed over time at a patient level to provide physicians and other health professionals a more complete picture of daily symptoms, medication efficacy and patient comfort.

The initial application of the electronic health journal is principally focused on pain and distress most commonly associated with oncology, some chronic conditions and post surgical patient populations. However, it will be appreciated that the same core concept can apply to various health related conditions or situations such as medication compliance, asthma management, depression, anxiety, etc.

Medical and health care providers are faced with a competitive environment in which they must constantly maintain or improve profitability and simultaneously improve patient care. Several factors contribute to the ever increasing costs of health care, whether it is delivered to the patient in a hospital, out-patient clinic setting or as part of in-home care. Health care deliverers face increased complexity in the types of treatment and services available, but also must provide these complex treatments and services efficiently, placing a premium on the institution's ability to provide complex treatment while maintaining complete and detailed medical records for each patient. Additionally, to date, there have been no reliable means for simultaneously monitoring objective patient data while providing for the patient to input subjective observation data in real time. The instant invention provides such a monitoring device in the form of a patient interactive, electronic health journal.

It is also advantageous to have an electronic health journal that provides patient data to a health care institution or medical provider into an interrelated automated system to provide real-time feedback on the effects of therapeutic and other drugs to the patient and at the same time provide for subjective patient input. Such a system can enable more effective medication dosage monitoring and to simultaneously allow patient reaction, interaction and subjective input as part of the patient's medical history. The interrelated system can also provide patients, doctors, nurses and other care-givers with updated medical information remotely including but not limited to when an additional drug is required, or when a scheduled treatment may not be as effective as previously contemplated, and automatically update the patient's medical health record database each time a medication or other care is given.

Inaccurate recording of the administration of medications and their effect on patient well being results in less than optimal medical treatment. Inadequate management of medication effectiveness also results in a failure to provide an accurate profile in treating a particular illness.

There have been numerous largely unsuccessful attempts to address the problems of the known prior art, particularly as it relates to remotely monitoring vital signs. For example, U.S. Pat. No. 4,518,267 discloses an event-module for the measurement and study of times, intervals, period, time series and durations. It is a complex device recommended for the treatment of addicts, such as smokers, alcoholics and over-eaters. The device is meant to be a therapy support in assisting the patient to fight his habit, rather than a means of diagnosing or evaluating the incidence, severity, and other medical information pertaining to a medical condition such as, for one example, pain associated with angina. The device is meant to divert the thoughts of the patient by providing him with a game to play. The patient can also enter times and durations of sporting events which he watches, further to divert his attention from his addiction.

U.S. Pat. No. 4,686,624 discloses an apparatus for acquiring and processing data on the dietetics and/or health of a person. The apparatus contains alphanumeric keys for inputting information, window for displaying the information that was introduced and various indications relating to them on the basis of pre-programmed parameters, and a device for selectively refusing inputs. On the basis of the inputs a computer generates instructions for the user of the apparatus from a program, such as what kind of food to eat, or medications to take. The device of the present invention is intended for the purpose of accurately recording patient-generated information pertaining to self-recognized symptoms of a known or suspected disease to facilitate physician or investigator evaluation of the patient's medical condition or response to treatment, under the patient's normal conditions of living.

U.S. Pat. No. 4,653,022 discloses a portable electrocardiogram storing apparatus, a patient actuatable switch, a plurality of electrocardiogram memories, and means for selecting when the switch is actuated, one of the memories for storing the digital signal of an electrocardiogram. The storage also contains timing signals relative to when the cardiogram was taken and stored. The primary purpose of this device, however, is to store information about clinical signs, which may or may not be apparent to the patient.

Also known is a device under the name of Holter monitor. This is a simplified electro-cardiographic sensor which provides a single lead tracing over a prolonged period of time. Therefore, it provides a snapshot, as is the case with electrocardiograms. The Holter monitor is designed for recording the onset and frequency and duration of ischemic events, but these do not necessarily correlate with symptomatic angina. The ischemic phenomenon of “silent angina”, in which the electrocardiographic changes typical of angina are present but without all the symptoms of an angina attack, is well documented. In such cases electrocardiographic tracing has no corollary in any patient-sensed symptoms, i.e. the patient feels no pain or onset of attack. Furthermore, the Holter monitor does not provide a means for recording the severity of the attack. Another purpose of the Holter monitor is to record.

Other devices have also been provided to assist in pain management and/or patient health monitoring which include, for example, an early telemetry system described in U.S. Pat. No. 3,603,881. According to that invention short transmission distances to a building's wiring system are covered using VHF transmission. Physiologic data such as electrocardiographic (ECG) data is collected by a sensor and transmitted by a VHF transmitter to a fixed VHF receiver RF transmitter coupled to the wiring system in the building. An RF receiver demodulator monitor is coupled to the building's wiring system at the nurse's station for receiving the physiologic data for patient monitoring and/or data recording.

A similar telemetry system for monitoring ECTG signals is described in U.K. Patent Application No. 2 003 276 except that telephone connections are used in place of the building wiring and the system is also designed to collect blood pressure, pulse rate, respiratory rate and the like and to relate that information to the physician via the telephone connections.

Other early telemetry systems of the type described by Lewis in U.S. Pat. No. 3,943,918 and by Crovella et al. in U.S. Pat. No. 4,121,573 use telemetric techniques to transmit data from a sensor device attached to the patient's chest via RF to a radio telemetry receiver for display and/or recording as desired. S.S. Ng described yet another telemetry system for ECG monitoring in an article entitled “Microprocessor-based Telemetry System for ECG Monitoring,” IEEE/Ninth Annual Conference of the Engineering in Medicine and Biology Society, CH2513-0, pages 1492-93 (1987). Ng describes a system for providing continuous ECG monitoring and analysis by means of a PC AT via wireless link. In the Ng system, the patient requires a transmitter which is carried by the patient for sensing and transmitting the patient's ECG signal to a central base station via wireless link. At the base station, a receiver recovers the original ECG signal from a few patients simultaneously for display.

Each of the above-described telemetry systems is designed primarily for hospital use and includes relatively expensive sensor arrays and processing devices for real-time patient monitoring and diagnosis. The real-time monitoring is generally used in an “alarm” mode to capture events, rather than to collect data over a period of time to determine trends which might indicate a more gradual deterioration or improvement in the patient's condition or to predict a forthcoming event. Also, these systems require the patient to remain in close proximity to the base stations including the receivers.

Bom et al. describe a portable physiological data monitoring/alert system in U.S. Pat. Nos. 4,784,162; 4,827,943; 5,214,939; 5,348,008; 5,353,793; and 5,564,429 in which one or more patients wear sensor harnesses including a microprocessor which detects potentially life-threatening events and automatically calls a central base station via radiotelemetry using a radio modem link. In a home or alternate site configuration, communications between the base station and remote unit is by way of commercial telephone lines. Generally, the system automatically calls “911” or a similar emergency response service when an abnormality is detected by the ECG monitor. Unfortunately, the sensor harness is quite cumbersome and conspicuous and includes sensors for performing an alert function rather than data collection and analysis functions.

Segalowitz discloses a wireless vital signs monitoring system in U.S. Pat. Nos. 4,981,141; 5,168,874; 5,307,818; and 5,511,553 including a precordial strip patch including a multi-layer flexible structure for telemetering data by radio frequency or single wire to hardware recording apparatus and a display monitor. Microsensors and conductive contact elements (CCEs) are mounted on the strip patch so as to permit simultaneous and continuous detection, processing and transmission of 12-lead ECG, cardiac output, respiration rate, peripheral blood oximetry, temperature of the patient, and ECG fetal heart monitoring via a single wavelength of radio frequency transmission. While the precordial strip patch used by Segalowitz purportedly transmits vital signs data up to 50 meters, it requires a dual-stage operational amplifier chip, an encoder modulator chip, a wireless transmitter chip including an oscillator, and other costly components such as artificial intelligence software, sound and visual alarms, and a microprocessor. As a result, the precordial strip patch is relatively expensive to manufacture and operate. Also, as with the other telemetry systems noted above, the emphasis of Segalowitz is on real-time monitoring and alerting of medical personnel to immediate medical needs of the patient.

Platt et al. also disclose a sensor patch for wireless physiological monitoring of patients in U.S. Pat. No. 5,634,468. Platt et al. describe a sensor and system for monitoring ECG signals remotely from patients located in non-hospital sites. In the Platt system, a sensor patch containing sensing electrodes, signal processing circuitry and radio or infra-red transmission circuitry is attached to the patient's body and preferably worn for at least a week before its power supply is exhausted and the sensor patch is thrown away. A receiver at a primary site in the vicinity of the patient receives the data transmitted by the sensor patch and stores the sensed data. When the patient feels discomfort or concern, or if the portable unit sounds an alarm, the patient telephones the monitoring station and downloads the stored data from the portable unit via the standard voice telecommunications network. The downloaded ECG data is then monitored and analyzed at the monitoring station. The receiver in the proximity of the patient may be a portable unit carried around by the patient, where the portable unit includes a receiver, a processor for processing the received data to identify abnormalities, a memory for storing the sensed data, and circuitry for interfacing to a telephone line to send the ECG data signals to the monitoring station. The monitoring station decodes the received ECG signals and performs beat and rhythm analysis for classification of the ECG data. If an abnormal condition is discovered, medical personnel in the vicinity of the patient are contacted. While the system described by Platt et al. may collect ECG data from the patient and process it at a remote monitoring station, the data is only collected when the patient initiates the data download. Otherwise, data is lost once the memory in the portable unit is full. No mechanism is provided for continuously collecting data, at all times, in a way which requires no patient action.

Finally, U.S. Pat. No. 5,522,396, Langer et al. discloses a telemetry system for monitoring the heart of a patient in which a patient station includes telemetering apparatus for transmitting the outputs of patient electrodes to a tele-link unit connected to a monitoring station by telephone lines. As in the Platt et al. system, Langer et al. transmit ECG data to a central location. However, unlike the Platt et al. system, the Langer et al. system checks the ECO data for predetermined events and automatically calls the monitoring station when such events are detected. A similar telemetry system is described by Davis et al. in U.S. Pat. No. 5,544,661 which initiates a cellular phone link from the patient to the central monitoring location when an event is detected. As with the Platt et al. system, neither of these systems provides a mechanism for continuously collecting data without patient action and none provide a simple device in which a patient can supplement or input objective health monitoring data with subjective input.

Accordingly, a simple, portable electronic health journal is desired which collects certain vital signs data from a patient using an inexpensive device which permits the continuous monitoring of a patient's vital signs data either with or without patient interaction (i.e. active or passive monitoring). Also, a patient health management system is desired which permits the collected patient data to be used in the overall assessment of patient well being and the like. The present invention has been designed to meet these needs in the art.

### SUMMARY OF THE INVENTION

In one embodiment, the present invention is a system for creating an electronic health journal comprising: at least one sensor configured to monitor an objective physiological parameter of a user and generate signals indicative of the monitored objective physiological parameter; a mobile electronic device comprising: a receiver for receiving the signals indicative of the monitored objective physiological parameter that are generated by the at least one sensor; input means for entering information relating to a subjective physiological condition of the user; a timer configured to timestamp the information relating to the subjective physiological condition of the user that is inputted by the user and the signals indicative of the monitored objective physiological parameter; an internal memory device configured to store the information relating to the subjective physiological condition of the user and the signals indicative of the monitored objective physiological parameter as a data log; and a transmitter; a local base station; and a remote data store, wherein the local base station is configured to automatically receive the data log from the mobile electronic device upon the mobile electronic device coming into communication proximity with the local base station and wherein the local base station is configured to transmit the data log to the remote data store.

In another embodiment, the present invention is a method of creating an electronic health journal comprising: monitoring an objective physiological parameter of a user with at least one sensor; generating signals indicative of the monitored objective physiological parameter of the user with the at least one sensor; receiving the signals indicative of the monitored objective physiological parameter with a mobile electronic device; receiving information relating to a subjective physiological condition of the user via input means of the mobile electronic device; time stamping the information relating to the subjective physiological condition of the user and the signals indicative of the monitored objective physiological parameter with a timer of the mobile electronic device; storing the information relating to the subjective physiological condition of the user and the signals indicative of the monitored objective physiological parameter as a data log on an internal memory device of the mobile electronic device; automatically receiving the data log from the mobile electronic device with a local base station when the mobile electronic device comes into communication proximity with the local base station; and transmitting the data log from the local base station to a remote data store.

In yet another embodiment, the present invention is a system for creating an electronic health journal comprising: a sensor device comprising: at least one sensor configured to monitor an objective physiological parameter of a user and generate signals indicative of the monitored objective physiological parameter; a timer configured to timestamp the signals indicative of the monitored objective physiological parameter; an internal memory device configured to store the signals indicative of the monitored objective physiological parameter as a first data log; and a transmitter; a mobile electronic device comprising: input means for entering information relating to a subjective physiological condition of the user; a timer configured to timestamp the information relating to the subjective physiological condition of the user that is inputted by the user; an internal memory device configured to store the information relating to the subjective physiological condition of the user as a second data log; and a transmitter; a local base station configured to automatically receive and time synchronize the first and second data logs, the local base station time synchronizing the first and second data logs by automatically receiving a current time reading from each of the timers of the sensor device and the mobile electronic device during receipt of the first and second data logs; and a remote data store, wherein the local base station is further configured to transmit the first and second data logs to the remote data store.

In even yet another embodiment, the present invention is a method of creating an electronic health journal comprising: monitoring an objective physiological parameter of a user with at least one sensor of a sensor device; generating signals indicative of the monitored objective physiological parameter with the sensor device; time stamping the signals indicative of the monitored objective physiological parameter with a timer of the sensor device; storing the signals indicative of the monitored objective physiological parameter as a first data log on an internal memory device of the sensor device; receiving information relating to a subjective physiological condition of the user via input means of a mobile electronic device; time stamping the information relating to the subjective physiological condition of the user with a timer of the mobile electronic device; storing the information relating to the subjective physiological condition of the user as a second data log on an internal storage device in the mobile electronic device; automatically receiving the first and second data logs from the sensor device and the mobile electronic device with a local base station; time synchronizing the first and second data logs with the local base station, the local base station time synchronizing the first and second data logs by automatically receiving a current time reading from each of the timers of the sensor device and the mobile electronic device during receipt of the first and second data logs; and transmitting the first and second data logs from the local base station to a remote data store.

### BRIEF DESCRIPTION OF THE DRAWINGS

FIG. 1 is a side perspective view of one exemplary embodiment of the electronic health journal of the present invention.

FIG. 2 is a side perspective view of an alternate embodiment of the electronic health journal of the present invention.

FIG. 3 is an example of the screen display and user interface of one embodiment of the electronic health journal of the present invention.

FIG. 4 is an example of a screen display and user interface of an alternate embodiment of the electronic health journal of the present invention.

FIG. 5 illustrates one embodiment of the electronic health journal in communication with a local base station unit (in partial phantom view) provided in accordance with the invention.

FIG. 6 illustrates the electronic health journal and local base station unit of FIG. 5 in communication with a remote data repository provided in accordance with the invention.

FIG. 7 illustrates a general flow chart of operation the electronic health journal of the present invention.

### DETAILED DESCRIPTION OF THE INVENTION

Briefly and in general terms, the present invention provides a new and improved device and methods for monitoring and tracking the administration and effectiveness of medications taken by patients remotely and for recording and managing pain and pain treating medications. The device, referred to herein as an electronic health journal, also provides a means for real-time patient data input regarding the patient's medical condition through the use of pain scores, considered by many to be “the Fifth Vital Sign.” A pain scale measures a patient's pain intensity or other features and are based on self-report, observational (behavioral), or physiological data. The self-report is considered of primary value and near or real-time reporting is preferable as this avoids the problems associated with temporal recollection.

Pain is typically associated with a variety of different underlying illnesses or injuries and can be considered as being either acute or chronic. Chronic or intractable pain is often endured over many years or decades. Long lasting chronic pain often occurs particularly in joints, in muscles, connective tissue and in the back. In the United States alone, chronic pain causes a loss of over 300 million working days per year. A patient is considered to have chronic pain when complaints thereof last longer than six months and over the course of time, chronic pain may form its own independent clinical syndrome. Patients suffering from chronic pain frequently develop psychological problems which can in turn lead to depression, concomitant pain relief substance abuse and in the worst cases, deeper depression and even attempted suicide.

In one embodiment, the electronic health journal comprises at least one local CPU having a variety of input and output devices for inputting patient data and/or for generating patient medical reports. An associated system of software programs operates either locally embedded on the device and/or on a local CPU to record, process, and send reports to one or more remote databases whose data is representative of the objective and subjective medical condition of a patient. Patient data may be stored locally until such time that it can be transmitted to a central repository. At the receiving end of the patient data stream, there are typically other CPUs connected together, along with at least one dedicated file server, to form an integrated network. Patient data is thus, inputted and stored locally by patient, sent to one or more remote CPUs, such as a remote computer, and is ultimately stored in a data storage device connected to the file server where it can be later retrieved and analyzed by medical professionals or specialized software.

More specifically, in a more detailed aspect by way of example and not necessarily of limitation, the electronic health journal includes a portable patient input device, a local CPU preferably also including a video display means and a data transmission CPU connected to and forming a local network. Additionally, the in operation, the electronic health journal constantly updates the health care, well-being database of information relating to the patient, the patient's condition, and the course of treatment prescribed to treat the patient's illness.

In one embodiment, the patient wears or carries the electronic health journal in the form of a portable input device including a NFC or Bluetooth module that can be read a local device reader using the corresponding appropriate communication protocol. During the course of monitoring of the medical condition, for example, chronic pain, the patient inputs subjective vital sign or other similar information which may in turn be transmitted to a local CPU or directly to a central repository.

The journal contains a CPU (microprocessor) that will control the journals behavior and detect when an input of data is made, store it in a local memory in the journal and transmit the data to a local device reader. The local device reader may subsequently transmit the data to a local or central repository.

A patient management system compares the patient's identity with a patient profile that preferably includes medication history and verifies that it is the correct profile for the patient. The patient's profile and identity can thus be stored in the database and linked to the treatment given to the patient to ensure complete and accurate tracking of all treatment given to the patient and its effect on pain management.

In one aspect of the invention, the electronic health journal that is provided is passive in nature. That is, the system operates to automatically detect and identify an individual, such as a patient without any particular action being required on the part of the individual. In another aspect, a passive RF transponder may be used in the electronic health journal which automatically detects patient activity and may further provide periodic prompts or queues to remind the patient to input subjective pain assessment.

In a further aspect, the electronic health journal also includes the capability of recording the physical location and movement of the patient via GPS, and maintains a history of the patient movement in a location event database. This database may also include a history of an electronic health journal's maintenance and calibration.

In another aspect, the electronic health journal includes the ability to track and report medication consumption and coordinate prescription refilling with the patient's heal care provider and an associated prescription fulfillment provider such as a hospital or on-line pharmacy. This assists in managing the inventory of medicines to ensure that medications are always available. A further advantage is that it enables a medical institution's administration to project supply usage and thus purchase supplies in quantities that ensure cost discounts without incurring excessive inventory carrying costs.

In yet other aspects, the electronic health journal employs RF (radio frequency) transmitters and receivers to connect the electronic health journal to other hardware elements such as blood pressure monitors, glucometers, respirometers and other data sending enabled devices capable of monitoring vital signs of a patient to form, essentially, an ad-hoc local area health network. This aspect is advantageous in that it provides increased patient health care and vital sign monitoring information while eliminating the need for costly network wiring throughout a patient domicile.

These and other advantages of the invention will become apparent from the following more detailed description when taken in conjunction with the accompanying drawings of illustrative embodiments.

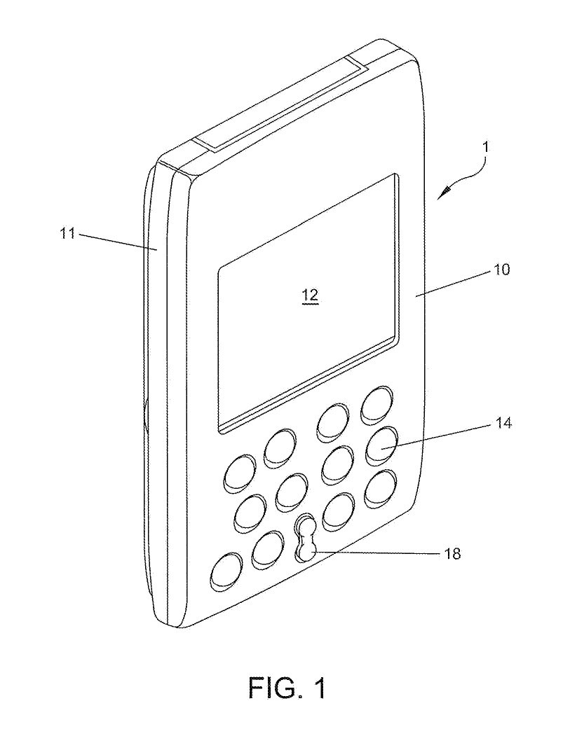

Referring to FIG. 1, one exemplary embodiment of the electronic journal 1 of the present invention having a housing 10, display 12 positioned on a front face of the housing, and input keys 14 positioned adjacent display 12 is shown. Housing 10 includes peripheral sidewalls 11 and a back surface (not shown) which may also include a clip or securing device to enable a patient to wear or otherwise carry the electronic health journal on their clothing so that it will be conveniently accessible by a patient. Positioned within housing 10 are a central processing unit (CPU), a data storage module, power supply, data transmission module and associated antenna for sending and receiving data. The power supply for the embodiment shown can include replaceable and/or chargeable batteries.

In the embodiment shown the electronic health journal is compact and portable having roughly the same dimensions as, for example, a typical cellular phone or smaller. Preferably, it is also light weight, durable, water proof or water resistant and rugged enough to with stand impact from accidental dropping. Display 12 can comprise a conventional LCD screen, a LED array, liquid crystal, numeric display or electroluminescent displays as well as LED lights arranged in a prearranged manner on a field comprising the general area of display 12. The key feature of display 12 is that it provides an interactive interface with a patient to enable the patient to input subjective self-reporting data regarding a particular health state, such as pain perception. In some preferred embodiments display 12 can also provide confirmatory feedback on successful data entry made by a patient, while in other preferred embodiments an electrical signal in the form of a beep tone or vibration may provide the data entry feedback.

In an alternate embodiment, the processing and interface of the electronic health journal described above with respect to FIG. 1 is provided in the form of an application for a mobile communication device. In one embodiment, the user downloads the electronic health journal application to their existing mobile communication device. The user's existing mobile communication device would then serve as the hardware described above with respect to FIG. 1 and the application would serve as the software for operating the electronic health journal.

It will be appreciated that the CPU, data storage module, data transmission module and associated antenna for sending and receiving data may be fabricated on a single dedicated chip or processor which can be programmed to perform the data entry, storage and transmission functions generally described herein. It will likewise be appreciated that certain desirable features such as event calendar, date and time stamp functions will also be part of the chip programming. In certain embodiments where GPS or other tracking methodologies are employed supplemental modules may be included within housing 10. Additionally, electronic health journal 1 may also include a device locator in the form of a distinct audible signal to assist a patient in finding a misplaced device within proximity of an associated base station, discussed below. Lastly, it will also be appreciated that the electronic health journal of the present invention may also be embodied in software or firmware resident on a conventional cellular telephone or PDA device adapted in accordance with the present invention.

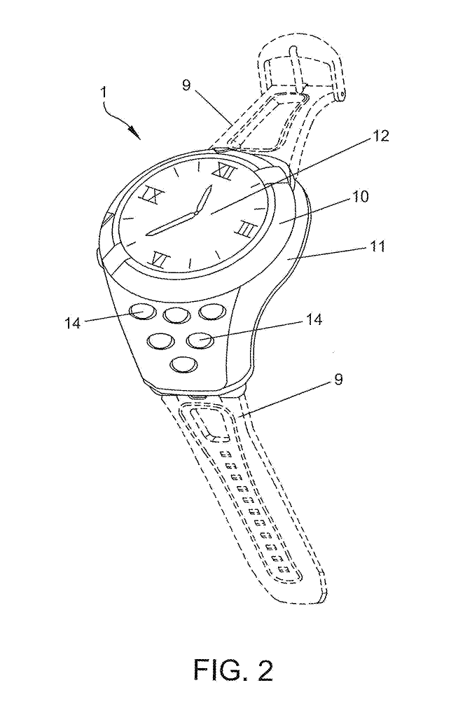

Referring to FIG. 2, an alternate preferred embodiment of the electronic health journal of the present invention shown in the form of a wrist watch or bracelet is illustrated. In this embodiment, the electronic health journal 1 and its internal components are miniaturized so that the electronic health journal can be conveniently and relatively inconspicuously worn by a patient. As shown in FIG. 2, housing 10 is secured to or may be an integral part of band 9. Housing 10 further includes peripheral sidewall 11 in the shape of a generally rectangular or circular watch or time piece defining on its upper surface a more or less transparent display area and which may further include the normal features and functions of a watch as a time piece. In this embodiment, display mat 12 comprises a LED array, thin-film or LCD display for displaying patient data above or as part of the watch face display. In yet other embodiments, display 12 may be preprogrammed to include a series of preset images generally corresponding to the Wong-Baker face pain scale or other sliding scale representing pain assessment. Positioned within housing 10 are the CPU, data storage module, power supply, data transmission module and associated antenna for sending and receiving data as generally described with respect to FIG. 1. The power supply for the embodiment shown can include replaceable and/or chargeable batteries, solar energy cells or so-called kinetic chargers which can generate and store electrical energy through normal body motion of the wearer.

In the preferred embodiment the system for creating an electronic health journal comprises a mobile electronic health journal device, at least one external sensor connected to the mobile electronic health journal device, a local base station, and a remote data store.

In the preferred embodiment, an external sensor is connected to the mobile electronic health journal. The external sensor is configured to monitor an objective physiological parameter of the user, generate signals indicative of the monitored objective physiological parameter, and transmit the signals to the mobile electronic health journal. An external sensor includes, among other things a blood pressure monitor, a heart rate monitor, a glucose meter, a thermometer and a respiratory meter. In alternate embodiments, there are more than one external sensors connected to the mobile electronic health journal, each external sensor monitoring different objective physiological parameters.

In the preferred embodiment, the mobile electronic health journal device comprises a receiver, user input means, an internal timer, internal memory, and a transmitter. The receiver is configured to receive the signals indicative of the monitored objective physiological parameter from the at least one external sensor. In an alternate embodiment, the receiver and transmitter are replaced with a transceiver.

The input means of the mobile electronic health journal are configured for the user to enter information relating to a subjective physiological condition. A subjective physiological condition includes, but is not limited to pain, depression, anxiety, irritability, drowsiness, dizziness, sneezing, dry mouth, weakness, headache, memory lapse, nausea, vomiting, sweating, constipation, itching, nightmares, visual distortion, heart palpitations, and muddled thinking. In the preferred embodiment, the user can enter information relating to any number of subjective physiological conditions into the mobile electronic health journal device. In one embodiment, the electronic health journal periodically prompts the user to input information relating to a subjective physiological condition.

In the preferred embodiment, the mobile electronic health journal device further comprises a timer. The timer is configured to timestamp the information relating to the entered subjective physiological condition and the received signals indicative of the monitored objective physiological parameter. Therefore, the timer of the mobile electronic health journal device timestamps both the subjective physiological condition and objective physiological condition received from the user.

In the preferred embodiment, after the user enters information relating to a subjective physiological condition or an objective physiological condition, the information is saved in a data log on the internal memory device of the mobile electronic health journal device. The data log correlates the information relating to the subjective physiological conditions and the objective physiological conditions of the user based on the times they were received. Therefore, after multiple entries of information regarding both subjective and objective physiological conditions, the data log will comprise all the entries in the order they were received by the mobile electronic health journal.

In either of the embodiments shown in FIGS. 1 and 2, it will be understood that patient data may be transmitted directly or indirectly via a local base station to a remote central repository such as a physician's or other medical provider's office. Preferably patient data, which includes pain assessment data among other things, is stored on the electronic health journal and then transmitted wirelessly to the central repository automatically upon the occurrence of one or more of the following events, a) at timed intervals; b) when there is a change in patient reported pain level; or c) when the electronic health journal comes into proximity with the local base station or NFC reader.

In the preferred embodiment, the mobile electronic health journal will transmit the data log to a local base station. The local base station is preferably a stationary CPU that the user would keep in their homes, offices, and other places where they are frequently present. The local base station is configured to connect to both the mobile electronic health journal and a remote data store. The local base station can connect to the mobile electronic using, among others, IrDA, Wi-Fi, NFC or Bluetooth. The local base station is further configured to receive the data log from the mobile electronic health journal and transmit it to the remote data store. In the preferred embodiment, the local base station only receives new, not previously received information from the mobile electronic health journal. Further, the local base station only transmits newly acquired information in the data log to the remote data store.

In one embodiment, the mobile electronic health journal periodically transmits an identification signal. Upon the mobile electronic health journal coming into communication proximity with the local base station, the local base station receives the identification signal and transmits a proximity signal to the mobile electronic device. Assuming the mobile electronic health journal is still in communication proximity with the local base station, the mobile electronic health journal receives the proximity signal and transmits the data log to the local base station. In the preferred embodiment, the mobile electronic health journal only transmits the identification signal when there is new, not previously transmitted, data within the data log.

In an alternate embodiment, the local base station periodically transmits an identification signal. Upon the mobile electronic health journal coming into communication proximity with the local base station, the mobile electronic health journal receives the identification signal and transmits the data log to the local base station. In the preferred embodiment, the mobile electronic health journal only transmits the data log to the local base station if there is new, not previously transmitted, data in the data log.

In another alternate embodiment, the sensor device comprises an internal memory device configured to store the signals indicative of the monitored objective physiological parameter as a data log. In this embodiment, the mobile electronic health journal also comprises an internal memory device for storing the information relating to the subjective physiological condition of the user in a second data log. Further, in this embodiment there is no means of communication between the sensor device and the mobile electronic health journal. Therefore, the local base station receives both the data log from the sensor device and the mobile electronic health journal. Preferably, the local base station time synchronizes both data logs to compensate for any differences in the internal timers of the sensor device and mobile electronic health journal. The local base station time synchronizes the data logs by acquiring the specifications of the timers of both the sensor device and mobile electronic device, comparing the timers, and compensating for any differences between the two timers. After synchronizing the two data logs, the local base station transmits the data logs to a remote data store. In an alternate embodiment, the local base station combines the two data logs after time synchronization but before transmission to the remote data store, thereby transmitting only one complete data log to the remote data store.

In the preferred embodiment, the system for creating the electronic health journal further comprises a remote data store. A data store can be any one of flash memory, an internal hard drive, an external database, a remote server, or any other data store known in the art. The local base station if configured to transmit the data log to the remote data store. The transmission of the data log to the data store can be done on an automatic or manually basis. Preferably, the local base station only transmits those portions of the data log that had not been previously transmitted to the data store. Upon receiving the data log, a doctor, physician, or other medical personnel can review the information within the data log and respond accordingly.

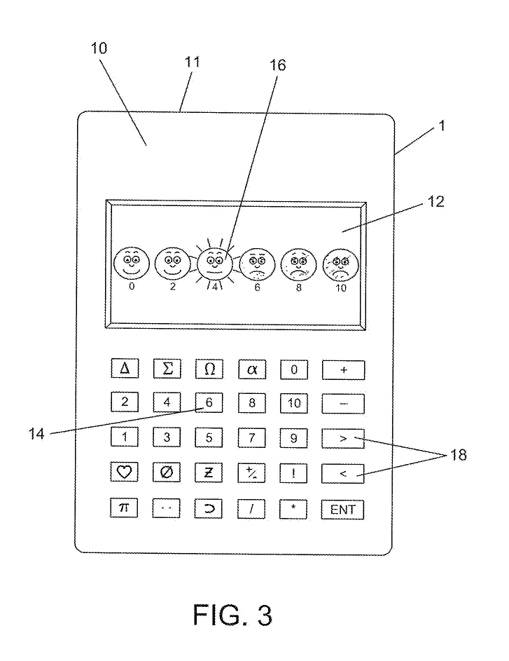

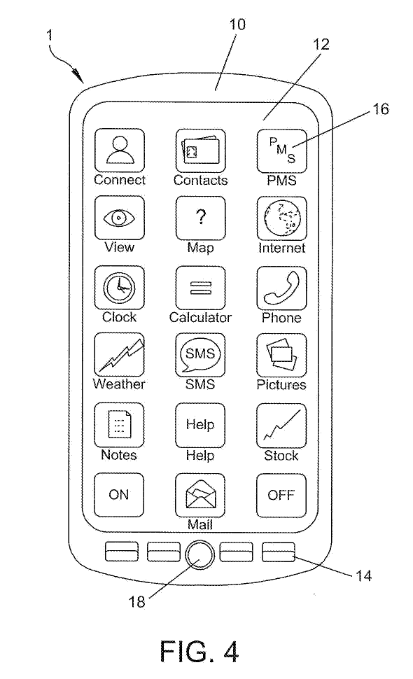

Referring to FIGS. 3 and 4, examples of screen displays and user interfaces in embodiments of the electronic health journal of the present invention are illustrated. Referring to FIG. 3, screen display 12 is part of a user interface including input module 16 comprising at least one input means such as selector 18 by which a user may enter information, including subjective physiological conditions, from either a menu of preselected choices or a user selected parameter. For example, if the subjective physiological condition is pain and the scale is the Wong Baker face pain scale, the Wong Baker face images are preprogrammed within a series of screen icon images and the user can scroll through the images and select the face choice that most closely approximates their pain assessment.

In an alternate embodiment, the user interface comprises content for gathering the subjective physiological condition of the user. The content for gathering the subjective physiological condition of the user can include pictures, questions, diagrams, scroll bars, and any other user interface known in the art. The content is used for the user to enter information regarding a subjective physiological condition.

In another alternate embodiment, the mobile electronic health journal is loaded with a patient profile. The patient profile comprises information relating to the user's medical history and currently prescribed medications. The patient profile is preferably stored on the internal memory of the mobile electronic health journal. The content of the user interface for gathering the subjective physiological conditions of the user is based on the patient profile. For example, the types of questions that might be asked of a patient vary depending on the types of medication they are currently prescribed and their past medical history. Further, the specific external sensors that are required might change depending on the medical history and/or medication of the patient. Therefore, the mobile electronic health journal adapts to the specific conditions of its individual patient.

In the preferred embodiment, the patient profile is loaded onto the mobile electronic health journal automatically from the remote data store through the local base station. In alternate embodiments, the patient profile can be loaded manually, or directly from the remote data store. Further, the patient profile can be updated on a routine basis to stay current with any changes in the user's medical history or prescribed medication.

In an alternate embodiment, the user enters medication consumption information into the mobile electronic device. The medication consumption information is then stored on the internal memory device as part of the data log and later transmitted to the local base station and ultimately the remote data store. In another alternate embodiment, the medication consumption information is used to coordinate prescription refilling with a local health care provided based on the currently prescribed medications in the patient profile and the user entered medication consumption information.

In other embodiments, such as that shown in FIG. 4 embedded programs that may include narrative descriptions, numeric scales or numbers referring to the subjective physiological condition can be used as either menu choices or data entries from which a user may select.

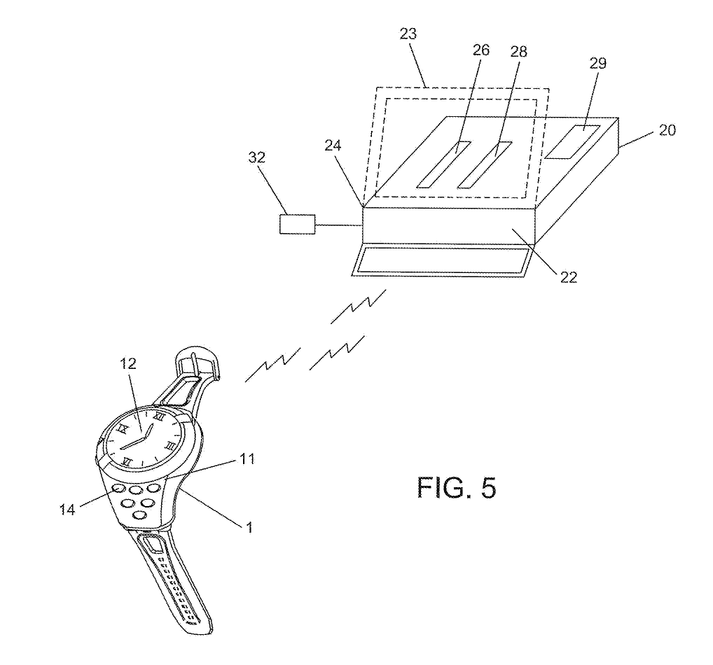

Referring to FIGS. 2 and 5, an alternate embodiment of the electronic health journal is illustrated. In the alternate embodiment, the electronic health journal takes the form of a watch or bracelet, given the limited real estate space of display 12, a patient may have fewer options in terms of parameter variables and numeric entries where 1=“no pain” to 7=“severe pain” may be most conveniently employed in the form of selector 18. It will be understood that screen display 12 may take other forms and that a variety of subjective physiological condition measurement scales may be successfully employed within the scope of the invention herein.

Additionally, the invention is not limited by either portable cellular phone or PDA sized devices or wrist watch and/or bracelet type embodiments. Specifically, the invention here is not limited in size or form and the electronic health journal may take other forms including but not limited to physical devices such as electronic check lists, pendants, fobs, pill boxes, medication dispensing containers, Blue-tooth enabled devices as well as computer software, hardware, firmware and computer icons through which a patient may enter data pertaining to a medical condition which is passed on to a medical provider or central repository.

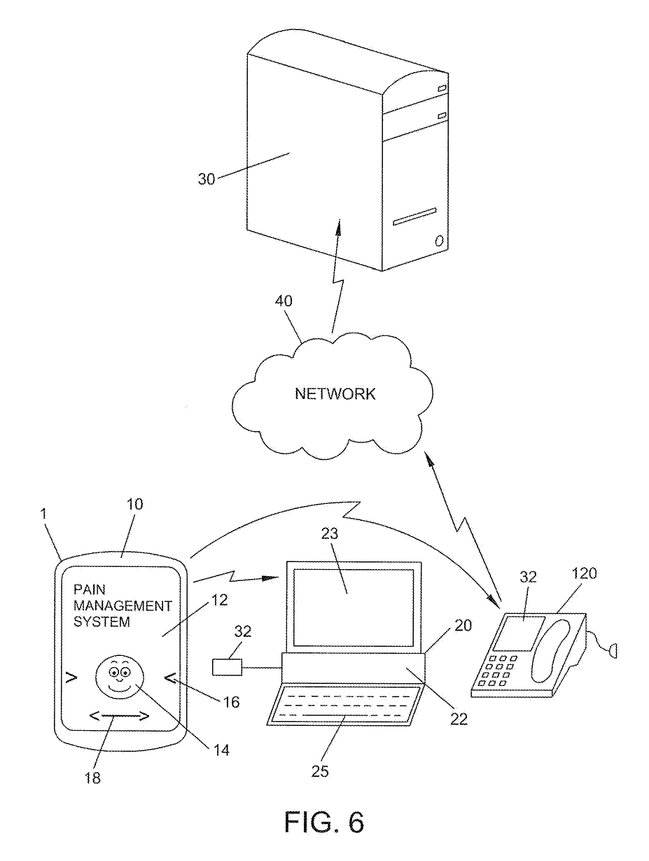

Referring to FIGS. 5 and 6, embodiments of the electronic health journal in communication with a local base station unit and a remote data store in accordance with the present invention are illustrated. Patient data is transmitted from electronic health journal 1 to local base station 20 which may be in the form of a personal computer or micro controller enabled device and which includes patient data receiving modules 22, typically associated with a NFC reader, screen 23, local data storage memory 24, transceiver module 26, communications gateway 28 and power supply 29. Local base station 20 may be a general purpose personal computer or a dedicated communications device for transmitting and receiving data to and from the electronic health journal and to and from remote repository 30. Communications gateway provides a data interchange link between local base station 20 and remote repository 30 where patient data may be stored and analyzed by one or more healthcare providers. Preferably, local base station data receiving modules 22 are programmed to automatically receive patient data at timed intervals, when there is a change in patient reported pain level and/or when electronic health journal 1 comes into proximity with the local base station or NFC reader.

In other embodiments, local base station 20 may also include a recharging device for electronic health journal 1 and may optionally include a cradle or electronic connection port 32 through which data exchange and initial and/or follow-up programming of electronic health journal 1 may be made. Local base station may also take the form of a conventional or specially adapted wired or wireless telephone 120.

It will be understood that local base station may communicate with remote repository 30 via any number of wired or wireless communication protocols including but not limited to conventional POTs, PTSN, dedicated hard wiring, internet access, and wireless telecommunication protocols, Wi-Fi and VoIP, by way of example. Remote repository 30 can be part of or transmit the data to network 40 or it may be a dedicated CPU assigned to a particular patient or group of patients. It will be understood that patient medical data stored by remote repository 30 and/or as part of data network 40 is stored securely and may be encrypted to ensure further security and to maintain patient confidentiality. Preferably, patient data may however be accessible globally via the internet or other global communications means so that valuable patient data and associated health records can be accessed by healthcare providers throughout the world using appropriate access means including access codes and passwords and the like. In one embodiment, electronic health journal 1 will include patient specific identification data which together with the required access codes which may be either resident on the electronic health journal or which may be entered by a patient via input keys 14, a healthcare provider will be able to access the complete medical history and records of a patient, including specifically, a patient's pain profile.

In still other embodiments, it will be appreciated that the local base station 20 may take the form of a local CPU connected to the internet or other suitable network or it may take the form of a wired or wireless telephone 120 which can transfer patient data to the appropriate receiving entity such as a medical provider or medical record repository for later retrieval and analysis. It will also be understood that in still other embodiments the local base station may be omitted entirely where the electronic health journal is adapted with sufficient communication capabilities.

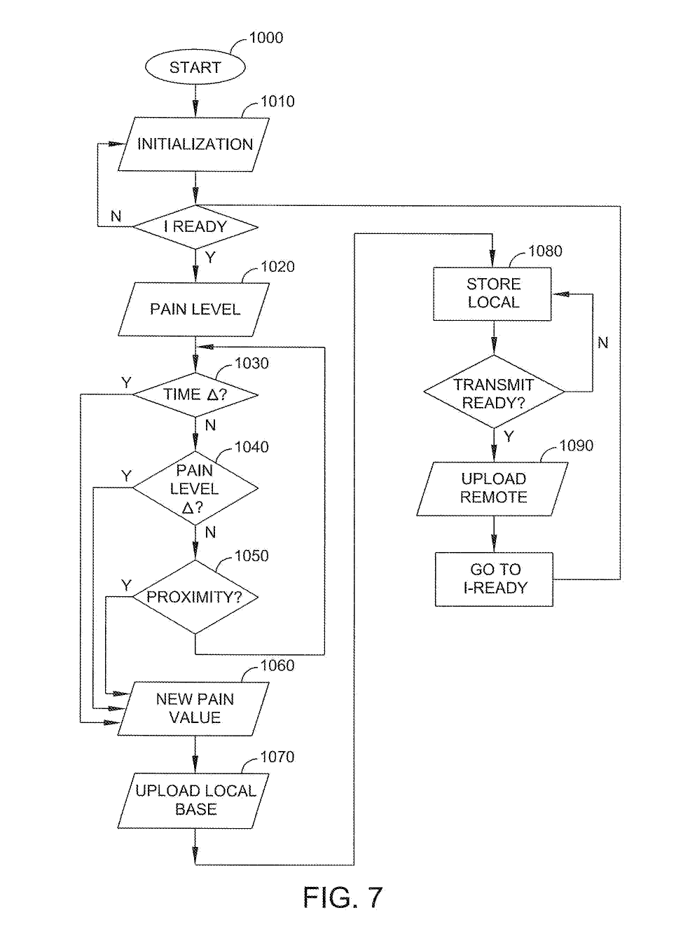

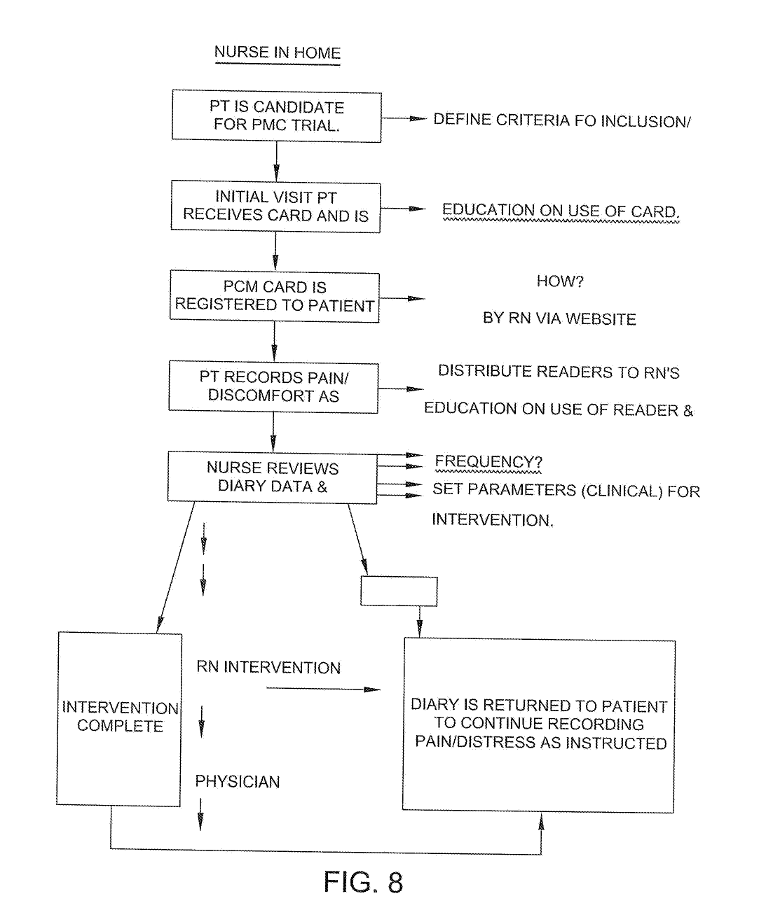

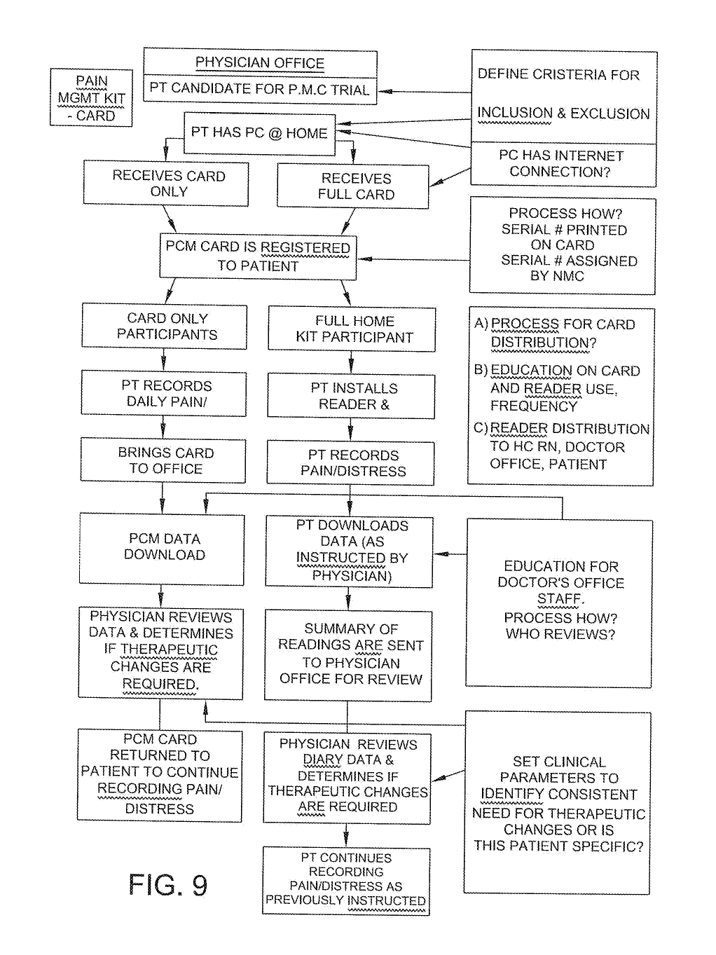

Referring to FIG. 7, a flow chart of operation of one embodiment of the electronic health journal of the present invention, particularly as it relates to pain management, is illustrated. The electronic health journal is initialized at start 1000. The initialization typically occurs in connection with a visit to a healthcare provider. Included in the initialization step is patient identification, calendar and medical history data initialization. In step 1010, base line pain level for a patient is entered and electronically date and time stamped, whereupon the electronic health journal internally stores a first values T1 and V1. V1 may be set by the healthcare provider or by the patient at the patient's convenience and preferably still at or near the location of an associated local base station; T1 is the initial time value corresponding to the date and time of V1. At step 1020 the initial values for T1 and V1 are stored in a memory queue for further processing.

Steps 1030 through 1050, relate to event occurrences and auto-processing of patient data at timed intervals (1030), change in patient reported pain level (1040) and/or when the electronic health journal comes into proximity with the local base station or NFC reader (1050). Step 1030 is typically a regular time interval represented by Tnew=T1+Tn where T1 is the initial time setting, Tn is a fixed number ranging from 1 to 24 hours, or by a change in the value of Vn, and Tnew is the new time value, stored with the new Vn in memory on the electronic health journal. V1 is the initial value of Vn, representing the base-line data for initial pain assessment. Any change in Vn will cause the new value to be reported together with the new time value. As the value of Tnew changes the data is similarly recorded in memory with the current Vn value. At step 1060, all patient data including stored values for both Tnew and Vn are automatically uploaded to the local base station at step 1070, where they are stored locally (step 1080) and then transmitted to a remote repository at step 1090. Upon successful transmittal and confirmation at step 1100, the process repeats and returns to step 1020 where the last set of patient data and time and pain values are placed back into memory to complete the loop.

It will be appreciated that the initial programming of the electronic health journal can occur in f different scenarios—one is where the patient is being seen by a nurse in the home and the other is based on the patient being seen in a physician's office. For example, the work flow of the typical “in home” initialization and “in office” initialization may follow as shown in Tables 1 and 2 below.

While the foregoing description and drawings represent the exemplary embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, sizes, and with other elements, materials, and components, without departing from the spirit or essential characteristics thereof. One skilled in the art will appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims, and not limited to the foregoing description or embodiments.

What is claimed is:

1. A system for creating an electronic health journal comprising:
at least one sensor configured to monitor an objective physiological parameter of a user and generate signals indicative of the monitored objective physiological parameter;
a mobile electronic device comprising:
a receiver for receiving the signals indicative of the monitored objective physiological parameter that are generated by the at least one sensor;
input means for entering information relating to a subjective physiological condition of the user;
a timer configured to timestamp the information relating to the subjective physiological condition of the user that is inputted by the user and the signals indicative of the monitored objective physiological parameter;
an internal memory device configured to store the information relating to the subjective physiological condition of the user and the signals indicative of the monitored objective physiological parameter as a data log; and
a transmitter;
a local base station; and
a remote data store, wherein the local base station is configured to automatically receive the data log from the mobile electronic device upon the mobile electronic device coming into communication proximity with the local base station and wherein the local base station is configured to transmit the data log to the remote data store.

2. The system of claim 1 wherein the mobile electronic device periodically prompts the user to input a subjective physiological condition.

3. The system of claim 1 wherein the mobile electronic device is loaded with a patient profile, the patient profile comprising information relating to the user's medical history and currently prescribed medications and being stored on the internal memory device as part of the data log.

4. The system of claim 3 wherein the local base station automatically receives an updated patient profile from the remote data store, the local base station transmits the updated patient profile to the mobile electronic device, the mobile electronic device receives the updated patient profile and updates the user's medical history and currently prescribed medications in the data log.

5. The system of claim 3 wherein the user enters medication consumption information into the mobile electronic device, the medication consumption information being stored on the internal memory device as part of the data log.

6. The system of claim 5 wherein the local base station coordinates prescription refilling with a health care provider based on the currently prescribed medications in the patient profile and the user entered medication consumption information.

7. The system of claim 1 wherein the at least one sensor includes a blood pressure monitor, a heart rate monitor, a glucose meter, a thermometer or a respiratory meter.

8. The system of claim 1 wherein the subjective physiological condition of the user is at least one of pain, depression, anxiety, irritability, drowsiness, dizziness, sneezing, dry mouth, weakness, headache, memory lapse, nausea, vomiting, sweating, constipation, itching, nightmares, visual distortion, heart palpitations, or muddled thinking.

9. The system of claim 1 wherein the mobile electronic device further comprises a GPS processor to record a physical location and movement of the user, the physical location and movement of the user being stored in the internal memory device as part of the data log.

10. The system of claim 1 wherein the subjective physiological condition of the user is determined from a Wong-Baker face pain scale.

11. The system of claim 1 wherein the mobile electronic device transmits the data log to the local base station using IrDA, Wi-Fi, NFC or Bluetooth.

12. The system of claim 1 wherein the local base station automatically receiving the data log from the mobile electronic device comprises the mobile electronic device periodically transmitting an identification signal, the local base station receiving the identification signal when the mobile electronic device is in communication proximity to the local base station and transmitting a proximity signal to the mobile electronic device, the mobile electronic device receiving the proximity signal and transmitting the data log to the local base station, and the local base station receiving the data log.

13. The system of claim 1 wherein the local base station automatically receiving the data log from the mobile electronic device comprises the local base station periodically transmitting an identification signal, the mobile electronic device receiving the identification signal when the mobile electronic device is in communication proximity to the local base station and transmitting the data log to the local base station, and the local base station receiving the data log.

14. The system of claim 1 wherein the mobile electronic device further comprises a display device for displaying a user interface comprising content for gathering the subjective physiological condition of the user.

15. The system of claim 14 wherein the mobile electronic device is loaded with a patient profile, the patient profile comprising information relating to the user's medical history and currently prescribed medications and being stored on the internal memory device as part of the data log, and wherein the content for gathering the subjective physiological condition of the user is based on the patient profile.

16. The system of claim 15 wherein the local base station automatically receives an updated patient profile from the remote data store, the local base station transmits the updated patient profile to the mobile electronic device, the mobile electronic device receives the updated patient profile and updates the user's medical history and currently prescribed medications in the data log.

17. The system of claim 15 wherein the user enters medication consumption information into the mobile electronic device, the medication consumption information being stored on the internal memory device as part of the data log.

18. The system of claim 17 wherein the local base station coordinates prescription refilling with a health care provider based on the currently prescribed medications in the patient profile and the user entered medication consumption information.

19. A method of creating an electronic health journal comprising:
monitoring an objective physiological parameter of a user with at least one sensor;
generating signals indicative of the monitored objective physiological parameter of the user with the at least one sensor;
receiving the signals indicative of the monitored objective physiological parameter with a mobile electronic device;
receiving information relating to a subjective physiological condition of the user via input means of the mobile electronic device;
time stamping the information relating to the subjective physiological condition of the user and the signals indicative of the monitored objective physiological parameter with a timer of the mobile electronic device;
storing the information relating to the subjective physiological condition of the user and the signals indicative of the monitored objective physiological parameter as a data log on an internal memory device of the mobile electronic device;
automatically receiving the data log from the mobile electronic device with a local base station when the mobile electronic device comes into communication proximity with the local base station; and
transmitting the data log from the local base station to a remote data store.

20. The method of claim 19 further comprising periodically prompting the user to input a subjective physiological condition via the mobile electronic device.

21. The method of claim 19 further comprising loading the mobile electronic device with a patient profile, the patient profile comprising information relating to the user's medical history and currently prescribed medications, and storing the patient profile on the internal memory device as part of the data log.

22. The method of claim 21 further comprising automatically receiving an updated patient profile from the remote data store with the local base station, transmitting the updated patient profile from the local base station to the mobile electronic device, receiving the updated patient profile with the mobile electronic device, and updating the user's medical history and currently prescribed medications in the data log.

23. The method of claim 21 further comprising receiving medication consumption information from the user via the input means of the mobile electronic device, the medication consumption information being stored on the internal memory device as part of the data log.

24. The method of claim 23 further comprising coordinating prescription refilling with a health care provider through the local base station based on the currently prescribed medications in the patient profile and the received medication consumption information.

25. The method of claim 19 wherein the at least one sensor includes a blood pressure monitor, a heart rate monitor, a glucose meter, a thermometer or a respiratory meter.

26. The method of claim 19 wherein the subjective physiological condition of the user is at least one of pain, depression, anxiety, irritability, drowsiness, dizziness, sneezing, dry mouth, weakness, headache, memory lapse, nausea, vomiting, sweating, constipation, itching, nightmares, visual distortion, heart palpitations, or muddled thinking.

27. The method of claim 19 further comprising recording a physical location and movement of the user with a GPS processor of the mobile communication device, and storing the physical location and movement of the user in the internal memory device as part of the data log.

28. The method of claim 19 wherein the subjective physiological condition of the user is determined from a Wong-Baker face pain scale.

29. The method of claim 19 wherein transmitting the data log from the mobile electronic device to the local base station is done using IrDA, Wi-Fi, NFC or Bluetooth.

30. The method of claim 19 wherein automatically receiving the data log from the mobile electronic device with the local base station comprises periodically transmitting an identification signal with the mobile electronic device, receiving the identification signal with the local base station when the mobile electronic device is in communication proximity to the local base station, transmitting a proximity signal from the local base station to the mobile electronic device, receiving the proximity signal with the mobile electronic device, transmitting the data log from the mobile electronic device to the local base station, and receiving the data log with the local base station.

31. The method of claim 19 wherein automatically receiving the data log from the mobile electronic device with the local base station comprises periodically transmitting an identification signal with the local base station, receiving the identification signal with the mobile electronic device when the mobile electronic device is in communication proximity to the local base station, transmitting the data log from the mobile electronic device to the local base station, and receiving the data log with the local base station.

32. The method of claim 19 further comprising displaying a user interface on a display device of the mobile electronic device, the user interface comprising content for gathering the subjective physiological condition of the user.

33. The method of claim 32 further comprising loading the mobile electronic device with a patient profile, the patient profile comprising information relating to the user's medical history and currently prescribed medications, and storing the patient profile on the internal memory device as part of the data log, and wherein the content for gathering the subjective physiological condition of the user is based on the patient profile.

34. The method of claim 33 further comprising automatically receiving an updated patient profile from the remote data store with the local base station, transmitting the updated patient profile from the local base station to the mobile electronic device, receiving the updated patient profile with the mobile electronic device, and updating the user's medical history and currently prescribed medications in the data log.

35. The method of claim 33 further comprising receiving medication consumption information from the user via the input means of the mobile electronic device, the medication consumption information being stored on the internal memory device as part of the data log.

36. The method of claim 35 further comprising coordinating prescription refilling with a health care provider through the local base station based on the currently prescribed medications in the patient profile and the user entered medication consumption information.

37. A system for creating an electronic health journal comprising:
a sensor device comprising:
at least one sensor configured to monitor an objective physiological parameter of a user and generate signals indicative of the monitored objective physiological parameter;
a timer configured to timestamp the signals indicative of the monitored objective physiological parameter;
an internal memory device configured to store the signals indicative of the monitored objective physiological parameter as a first data log; and
a transmitter;
a mobile electronic device comprising:
input means for entering information relating to a subjective physiological condition of the user;
a timer configured to timestamp the information relating to the subjective physiological condition of the user that is inputted by the user;
an internal memory device configured to store the information relating to the subjective physiological condition of the user as a second data log; and
a transmitter;
a local base station configured to automatically receive and time synchronize the first and second data logs, the local base station time synchronizing the first and second data logs by automatically receiving a current time reading from each of the timers of the sensor device and the mobile electronic device during receipt of the first and second data logs; and
a remote data store, wherein the local base station is further configured to transmit the first and second data logs to the remote data store.

38. The system of claim 37 wherein the local base station is further configured to combine the first and second data logs after they have been time synchronized to create a complete data log and transmit the complete data log to the remote data store.

39. The system of claim 37 wherein the mobile electronic device periodically prompts the user to input a subjective physiological condition.

40. The system of claim 37 wherein the mobile electronic device is loaded with a patient profile, the patient profile comprising information relating to the user's medical history and currently prescribed medications and being stored on the internal memory device as part of the data log.

41. The system of claim 40 wherein the local base station automatically receives an updated patient profile from the remote data store, the local base station transmits the updated patient profile to the mobile electronic device, the mobile electronic device receives the updated patient profile and updates the user's medical history and currently prescribed medications in the data log.

42. The system of claim 40 wherein the user enters medication consumption information into the mobile electronic device, the medication consumption information being stored on the internal memory device as part of the data log.

43. The system of claim 42 wherein the local base station coordinates prescription refilling with a health care provider based on the currently prescribed medications in the patient profile and the user entered medication consumption information.

44. The system of claim 37 wherein the at least one sensor includes a blood pressure monitor, a heart rate monitor, a glucose meter, a thermometer or a respiratory meter.

45. The system of claim 37 wherein the subjective physiological condition of the user is at least one of pain, depression, anxiety, irritability, drowsiness, dizziness, sneezing, dry mouth, weakness, headache, memory lapse, nausea, vomiting, sweating, constipation, itching, nightmares, visual distortion, heart palpitations, or muddled thinking.

46. The system of claim 37 wherein the mobile electronic device further comprises a GPS processor to record a physical location and movement of the user, the physical location and movement of the user being stored in the internal memory device as part of the data log.

47. The system of claim 37 wherein the subjective physiological condition of the user is determined from a Wong-Baker face pain scale.

48. The system of claim 37 wherein the mobile electronic device transmits the data log to the local base station using IrDA, Wi-Fi, NFC or Bluetooth.

49. The system of claim 37 wherein the local base station automatically receiving the data log from the mobile electronic device comprises the mobile electronic device periodically transmitting an identification signal, the local base station receiving the identification signal when the mobile electronic device is in communication proximity to the local base station and transmitting a proximity signal to the mobile electronic device, the mobile electronic device receiving the proximity signal and transmitting the data log to the local base station, and the local base station receiving the data log.

50. The system of claim 37 wherein the local base station automatically receiving the data log from the mobile electronic device comprises the local base station periodically transmitting an identification signal, the mobile electronic device receiving the identification signal when the mobile electronic device is in communication proximity to the local base station and transmitting the data log to the local base station, and the local base station receiving the data log.

51. The system of claim 37 wherein the mobile electronic device further comprises a display device for displaying a user interface comprising content for gathering the subjective physiological condition of the user.

52. The system of claim 51 wherein the mobile electronic device is loaded with a patient profile, the patient profile comprising information relating to the user's medical history and currently prescribed medications and being stored on the internal memory device as part of the data log, and wherein the content for gathering the subjective physiological condition of the user is based on the patient profile.

53. The system of claim 52 wherein the local base station automatically receives an updated patient profile from the remote data store, the local base station transmits the updated patient profile to the mobile electronic device, the mobile electronic device receives the updated patient profile and updates the user's medical history and currently prescribed medications in the data log.

54. The system of claim 52 wherein the user enters medication consumption information into the mobile electronic device, the medication consumption information being stored on the internal memory device as part of the data log.

55. The system of claim 54 wherein the local base station coordinates prescription refilling with a health care provider based on the currently prescribed medications in the patient profile and the user entered medication consumption information.

56. A method of creating an electronic health journal comprising:
monitoring an objective physiological parameter of a user with at least one sensor of a sensor device;
generating signals indicative of the monitored objective physiological parameter with the sensor device;
time stamping the signals indicative of the monitored objective physiological parameter with a timer of the sensor device;
storing the signals indicative of the monitored objective physiological parameter as a first data log on an internal memory device of the sensor device
receiving information relating to a subjective physiological condition of the user via input means of a mobile electronic device;
time stamping the information relating to the subjective physiological condition of the user with a timer of the mobile electronic device;
storing the information relating to the subjective physiological condition of the user as a second data log on an internal storage device in the mobile electronic device;
automatically receiving the first and second data logs from the sensor device and the mobile electronic device with a local base station;
time synchronizing the first and second data logs with the local base station, the local base station time synchronizing the first and second data logs by automatically receiving a current time reading from each of the timers of the sensor device and the mobile electronic device during receipt of the first and second data logs; and
transmitting the first and second data logs from the local base station to a remote data store.

57. The method of claim 56 further comprising periodically prompting the user to input a subjective physiological condition via the mobile electronic device.

58. The method of claim 56 further comprising loading the mobile electronic device with a patient profile, the patient profile comprising information relating to the user's medical history and currently prescribed medications, and storing the patient profile on the internal memory device as part of the second data log.

59. The method of claim 58 further comprising automatically receiving an updated patient profile from the remote data store with the local base station, transmitting the updated patient profile from the local base station to the mobile electronic device, receiving the updated patient profile with the mobile electronic device, and updating the user's medical history and currently prescribed medications in the second data log.

60. The method of claim 58 further comprising receiving medication consumption information from the user via the input means of the mobile electronic device, the medication consumption information being stored on the internal memory device as part of the second data log.

61. The method of claim 60 further comprising coordinating prescription refilling with a health care provider through the local base station based on the currently prescribed medications in the patient profile and the received medication consumption information.

62. The method of claim 56 wherein the at least one sensor includes a blood pressure monitor, a heart rate monitor, a glucose meter, a thermometer or a respiratory meter.

63. The method of claim 56 wherein the subjective physiological condition of the user is at least one of pain, depression, anxiety, irritability, drowsiness, dizziness, sneezing, dry mouth, weakness, headache, memory lapse, nausea, vomiting, sweating, constipation, itching, nightmares, visual distortion, heart palpitations, or muddled thinking.

64. The method of claim 56 further comprising recording a physical location and movement of the user with a GPS processor of the mobile communication device, and storing the physical location and movement of the user in the internal memory device as part of the second data log.

65. The method of claim 56 wherein the subjective physiological condition of the user is determined from a Wong-Baker face pain scale.

66. The method of claim 56 wherein transmitting the data log from the mobile electronic device to the local base station is done using IrDA, Wi-Fi, NFC or Bluetooth.

67. The method of claim 56 wherein automatically receiving the first and/or second data logs from the sensor device and/or mobile electronic device with the local base station comprises periodically transmitting a first identification signal with the sensor device and a second identification signal with the mobile electronic device, receiving the first and/or second identification signals with the local base station when the sensor device and/or mobile electronic device is in communication proximity to the local base station, transmitting a proximity signal from the local base station to the sensor device and/or mobile electronic device, receiving the proximity signal with the sensor device and/or mobile electronic device, transmitting the first and/or second data logs from the sensor device and/or mobile electronic device to the local base station, and receiving the first and/or second data logs with the local base station.

68. The method of claim 56 wherein automatically receiving the first and/or second data logs from the sensor device and/or mobile electronic device with the local base station comprises periodically transmitting an identification signal with the local base station, receiving the identification signal with the sensor device and/or mobile electronic device when the sensor device and/or mobile electronic device is in communication proximity to the local base station, transmitting the first and/or second data logs from the sensor device and/or mobile electronic device to the local base station, and receiving the first and/or second data logs with the local base station.

69. The method of claim 56 further comprising displaying a user interface on a display device of the mobile electronic device, the user interface comprising content for gathering the subjective physiological condition of the user.

70. The method of claim 69 further comprising loading the mobile electronic device with a patient profile, the patient profile comprising information relating to the user's medical history and currently prescribed medications, and storing the patient profile on the internal memory device as part of the second data log, and wherein the content for gathering the subjective physiological condition of the user is based on the patient profile.

71. The method of claim 70 further comprising automatically receiving an updated patient profile from the remote data store with the local base station, transmitting the updated patient profile from the local base station to the mobile electronic device, receiving the updated patient profile with the mobile electronic device, and updating the user's medical history and currently prescribed medications in the second data log.

72. The method of claim 70 further comprising receiving medication consumption information from the user via the input means of the mobile electronic device, the medication consumption information being stored on the internal memory device as part of the second data log.

73. The method of claim 72 further comprising coordinating prescription refilling with a health care provider through the local base station based on the currently prescribed medications in the patient profile and the user entered medication consumption information.
